(19) United States
(12) Patent Application Publication
(10) Pub. No.: US 2024/0252710 A1
(43) Pub. Date: Aug. 1, 2024

(54) SYSTEM AND METHOD TO PRODUCE ATMOSPHERIC NUTRITIONAL AND DISINFECTANT IODINE

(71) Applicant: NOVO INTEGRATED SCIENCES INC., Bellevue, WA (US)

(72) Inventor: Terence Francis Mullins, Magog (CA)

(21) Appl. No.: 18/291,836

(22) PCT Filed: Jul. 25, 2022
(86) PCT No.: PCT/IB2022/056855

§ 371 Date: Jan. 24, 2024

Related U.S. Application Data
(60) Provisional application No. 63/226,644, filed on Jul. 28, 2021.

(51) Int. Cl. A61L 9/14; A61L 2/22; A61L 2/24; A61L 101/06

(52) U.S. Cl. CPC A61L 9/14; A61L 2/22; A61L 2/24; A61L 2101/06; A61L 2202/11; A61L 2202/14; A61L 2202/25; A61L 2209/11; A61L 2209/16

(57) ABSTRACT

A system and method for producing atmospheric gaseous iodine is disclosed. The system includes an airless reservoir for holding iodine, an iodine source connected to the reservoir for providing the iodine, a dispersal device for dispersing the iodine from the reservoir into an environment, and an electronic controller connected to the iodine source and the dispersal device for controlling the iodine source and the dispersal device. The method includes providing iodine from an iodine source to an iodine reservoir and dispersing the iodine from the reservoir into an atmosphere with an iodine dispersal device, wherein providing and dispersing the iodine is controlled by an electronic connecter.

### FIELD

The present disclosure relates to atmospheric gaseous iodine, and more particularly to a system and method for production and delivery of atmospheric gaseous iodine.

### BACKGROUND

To many, there is no distinction between iodine and iodides, however, the distinctions are critical. We consume iodides that are then converted to iodine within the body by the thyroid and some organs. We need both iodine and iodides in our body. Iodine is biocidal, Iodides are not. Iodine is unstable and difficult to contain and control without the body, whereas iodides are stable.

Iodine in its natural state is a gas. Elemental iodine is manufactured and sold in a solid form, flake or prilled. Elemental iodine is then used to produce many derivative products one being aqueous iodine.

A human adult inhales approximately 11 cubic meters of air a day. As humans, we can only breathe air within certain strict guidelines. For example, if air is too hot or too cold, breathing problems occur; chemical contaminants and pollutants carried in the air are harmful to us; and airborne organic and inorganic matter leads to allergic reactions.

Whether we heat or air condition our homes, we are altering ambient air quality. Air purifiers for residential and commercial uses have grown significantly over the past 40 years. They provide air movement, chemical contaminant removal, and in some cases point of treatment air disinfection. Humidifiers and dehumidifiers keep us in our comfort zones. Commercial and industrial buildings, for example hospitals and research laboratories, also require pre-determined air quality. Hospitals purchase costly state of the art air treatment systems for reduction of disease transmission.

Mullins (#US2016/0206768) utilized iodinated water through humidification to produce variable sized water particles containing iodine. This technology is restricted to using humidification processes to introduce iodine to the air and does not perform well in a humid environment. Many people, especially the elderly and people with existing breathing conditions, find it difficult to breathe in a humid environment. Therefore, this technology has restrictions under the conditions that it can operate and is not suitable for a universal designation. Also, the cost and size of this device is prohibitive for residential or commercial applications.

Iodophor/Povidone/Betadine (plastic iodine) are historical iodine technologies used in hospitals as surface and topical disinfectants. In 2020 a Connecticut University study used povidone to swab nostrils of human subjects and killed corona virus in less than 15 seconds. The only active biocide in these technologies is molecular iodine provided at very low levels. By design, these biocides have molecular structures that utillize inert scaffolding materials not suitable for human consumption or inhalation. Plastics, surfactants, and acids are among the inert materials.

Iodine is a required and essential human, animal and plant micronutrient. Methods of delivering iodine into our bodies have been by either ingestion or intravenous. Table salt has been the main carrier vehicle for over a century to obtain the minimum iodide requirements in our diets. Iodine in the form of iodide/iodate was added to salt at the turn of the 20th century to address iodine deficiency disorders globally. Iodine Supplementation is required as large areas of the world are iodine deficient. In the developed world, iodine deficiency has increased more than four-fold over the past 40 years. Nearly 74% of normal, “healthy” adults may no longer consume enough iodine (iodide). Health risks associated with salt consumption have reduced the consumption in developed countries and the effect is a lack of iodine in our diets. A human brain continues to grow until we are in our twenties. A large percentage of American children and teens have a poor diet that is lacking the most important essential micronutrient for cerebral growth and function, iodine.

The present invention addresses the issues by employing atmospheric iodine as a micronutrient, aerosol disinfectant and therapeutic treatment.

### SUMMARY

In the present disclosure, a system and method for producing atmospheric gaseous iodide is provided.

Thus, by one broad aspect of the present invention, a system is provided for for producing atmospheric gaseous iodine, the system comprising an airless reservoir for holding an iodine, an iodine source connected to the reservoir for providing the iodine to the reservoir, a dispersal device for dispersing the iodine from the reservoir into an atmosphere, and an electronic controller connected to the iodine source and the dispersal device for controlling the iodine source and the dispersal device.

By a further aspect of the present invention, a method is provided for producing atmospheric gaseous iodine, the method comprising providing iodine from an iodine source to an iodine reservoir and dispersing the iodine from the reservoir into an atmosphere with an iodine dispersal device, wherein providing and dispersing the iodine is controlled by an electronic connecter.

A further understanding of the functional and advantageous aspects of the invention can be realized by reference to the following detailed description and drawings.

### BRIEF DESCRIPTION OF THE DRAWINGS

Embodiments disclosed herein will be more fully understood from the following detailed description taken in connection with the accompanying drawings, which form a part of this application, and in which:

FIG. 1 is a schematic representation of a system for providing atmospheric gaseous iodine, according to an embodiment of the present disclosure.

FIG. 2 is an alternate embodiment of the system illustrated in FIG. 1, in which atmospheric gaseous iodine is dispersed in an air vent.

FIG. 3 is a further alternate embodiment of the system illustrated in FIG. 1, in which atmospheric gaseous iodine is dispersed in a heating, ventilation and air conditioning (HVAC) duct.

FIG. 4 is a schematic representation of a system for providing atmospheric gaseous iodine, according to an alternate embodiment of the present disclosure.

### DETAILED DESCRIPTION

The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

Although the invention has been described with a preferred embodiment, it should be noted that the inventor can make various modifications, additions and alterations to the invention without departing from the original scope as described in the present disclosure.

The present invention produces gaseous iodine enriched air. The content of gaseous iodine within a cubic meter of air can be varied based on application for disinfection, micronutrient, commercial and industrial requirements.

Atmospheric Iodine is fatal to microbes in the air and kills microbes on surfaces that the iodine enriched air within a room comes in contact with. Concentration levels of iodine enriched air within a room that are fatal to microbes are safe and healthy for humans to breathe.

A system is provided for producing atmospheric gaseous iodine. The system includes an airless reservoir for holding iodine, an iodine source connected to the reservoir for providing the iodine to the reservoir, a dispersal device for dispersing the iodine from the reservoir into an atmosphere, and an electronic controller connected to the iodine source and the dispersal device for controlling the iodine source and the dispersal device.

The iodine source provides either liquid or solid iodine, which is sublimated to produce gaseous iodine. The airless reservoir connected to the iodine source houses the gaseous iodine. The dispersal device disperses the gaseous iodine from the reservoir to the surrounding atmosphere or environment. The sublimation process is controlled by the electronic controller, which is capable of monitoring and adjusting the function of devices within the apparatus. Atmospheric gaseous iodine is introduced in a controlled fashion through the dispersal device into a given environment by the electronic controller. The invention allows for replaceable mated aqueous or solid iodine recharges of the iodine source to accommodate predetermined maintenance and replenishing schedules of the apparatus.

Iodine, in its natural gaseous state is infused into air by the system to provide for a recommended daily allowance iodine micronutrient uptake through inhalation, aerosol iodine disinfection or human health inhalation therapy. Iodine in its natural state is the only biocidal element infused into the air by the system.

In an embodiment of the system, the reservoir receives an airless aqueous iodine. A sonic frequency disk abuts the reservoir, for sonicating the airless aqueous iodine in the reservoir to produce gaseous iodine. The iodine source may include an airless aqueous iodine bladder and an iodine source peristaltic pump to transport the aqueous iodine from the bladder to the reservoir. The electronic controller is connected to the sonic frequency disk to regulate its function.

Aqueous iodine produces atmospheric iodine when the iodine is released from the water carrier molecule through specific sonic sublimation within a room or establishment. Atmospheric iodine has increased molecular mobility and action when unencumbered by a water molecule bond. The concentration of iodine produced within a cubic meter of air is controlled through a combination of sonic sublimation processes and the quantity and metering of aqueous iodine from a known volume reservoir.

In a further embodiment, the system may be sized for tabletop and/or wall mount applications within a single room or establishment. The system includes an iodine reservoir capable of receiving, containing and maintaining a concentrate of aqueous iodine either in a pure form or a tincture form and a self-priming peristaltic pump connecting the aqueous iodine source to the iodine reservoir. An iodine-inert liquid feed line connects the peristaltic pump to the reservoir. A frequency-specific sonic sublimation system breaks the molecular bond between the water and iodine. The sonic sublimation system is located within a spire, and includes a sonic vibrator abutting the reservoir, comprising a heated micro reservoir and an overflow tray inert to aqueous iodine. A micro fan is located beneath the sonic sublimation system within the spire. The spire has single direction flow incorporating a circular, oval or needle like air dispersal apparatus at the apex.

The system further includes the electronic controller, which is preprogrammed to receive information from the iodine reservoir, control the rate of aqueous iodine being introduced to the sonic system by the micro pump, control the frequency of the sonic system to produce atmospheric iodine, and control the operation of the micro fan within the system to disperse the gaseous iodine into a known volume of air within a room or establishment. The system can operate on a continuous or intermittent basis of gaseous iodine production. The electronic controller may include an interior clock to control and interact with the multiple devices within the apparatus to maintain operation and maintenance schedules. The electronic controller may further include an interactive screen located on the exterior of the apparatus for an operator to program operations.

Aqueous iodine at a predetermined concentration and volume is contained in a special canister with a collapsible bladder inert to iodine and a viton or kynar feed line to the micro pump. A self-priming micro peristaltic pump with flows ranging from zero to 30 ml per minute draws aqueous iodine from the iodine canister and delivers the aqueous iodine to the sonic reservoir.

The reservoir volume within the tabletop apparatus is approximately four millilitres and is of a column design whereby the column is of sufficient height and design as to reduce the splashing effect created by the sonic disk. The sonic reservoir can be fabricated from plastic inert to iodine or fabricated from stainless steel or glass.

In another embodiment of the invention the sonic reservoir is heated, thereby warming the aqueous iodine solution to increase the sublimation process. In this embodiment the heated sonic reservoir is capable of complete sublimation of the iodine within the aqueous solution. The sonic disk system can operate at lower agitation frequencies in combination with the heated sonic reservoir to increase the sublimation process. The sonic disk is utilized to rapidly humidify any pure water solution remaining within the sonic reservoir post iodine sublimation.

Sonic disks are a known technology with many different uses and different manufacturers. Sonic disks require a coating treatment process prior to being introduced to aqueous iodine. This treatment process allows for the proper function of the sonic disk and reduces the detrimental effects of iodine on the disks.

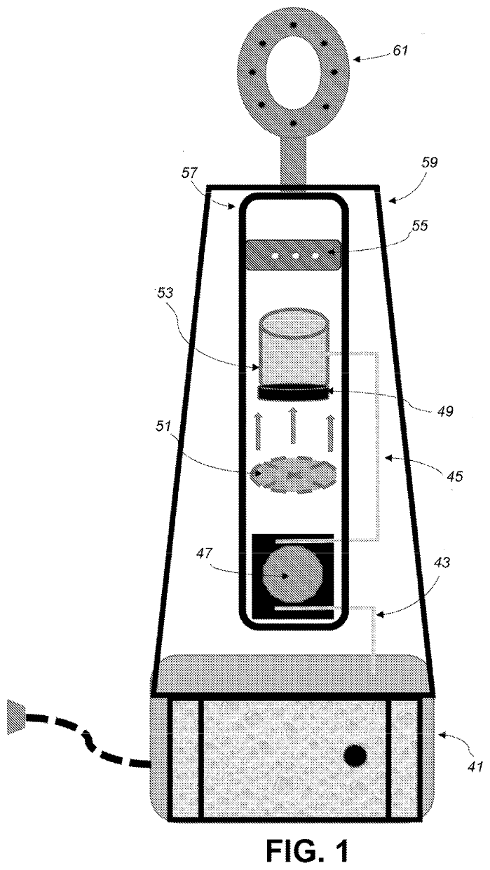

Referring now to FIG. 1, an embodiment of the apparatus and method for the production of atmospheric iodine is illustrated. In this embodiment, the iodine source is a replaceable iodine canister 100 located at the base of the apparatus. The peristaltic pump 200 is located above the iodine canister 100. The liquid feed supply line 120 is connected to the pump 200. The pump 200 meters liquid through feed line 121 to supply the iodine reservoir 250. The fan 300 is activated concurrently to the sonic disk 400 activation. Upon sonic disk activation, gaseous iodine is produced and exits the apparatus through the dispersal ring (550) in a room or establishment. The controller (888) is located on the exterior of the apparatus (50) and can be programmed to accommodate room dimensions and air turnover within the environment. The components are contained within the apparatus housing on a spire component mounting rack 600.

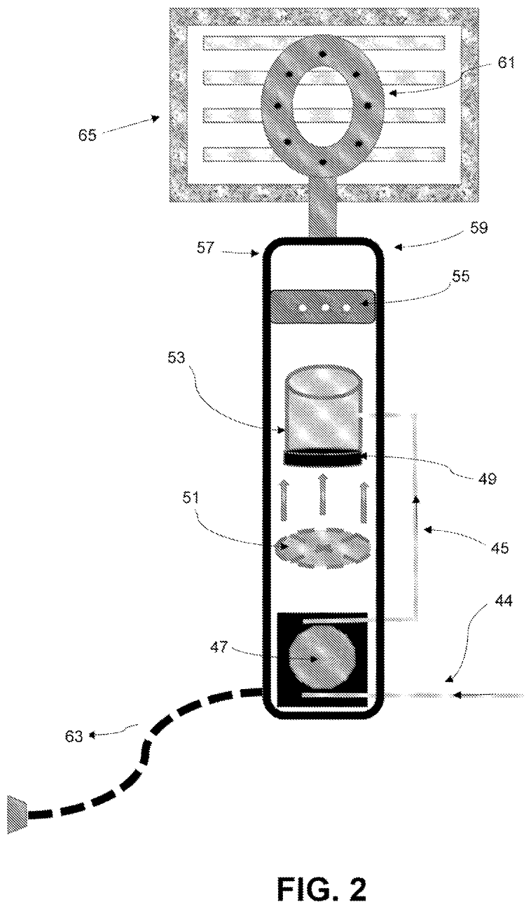

Referring to FIG. 2, a schematic flow diagram illustrates an alternative apparatus and method for providing atmospheric iodine to multiple rooms. The spire mounting rack is mounted on a wall in front of a fresh air return vent. In this example, the spire mounting rack is connected to a remote source aqueous iodine supply ##.

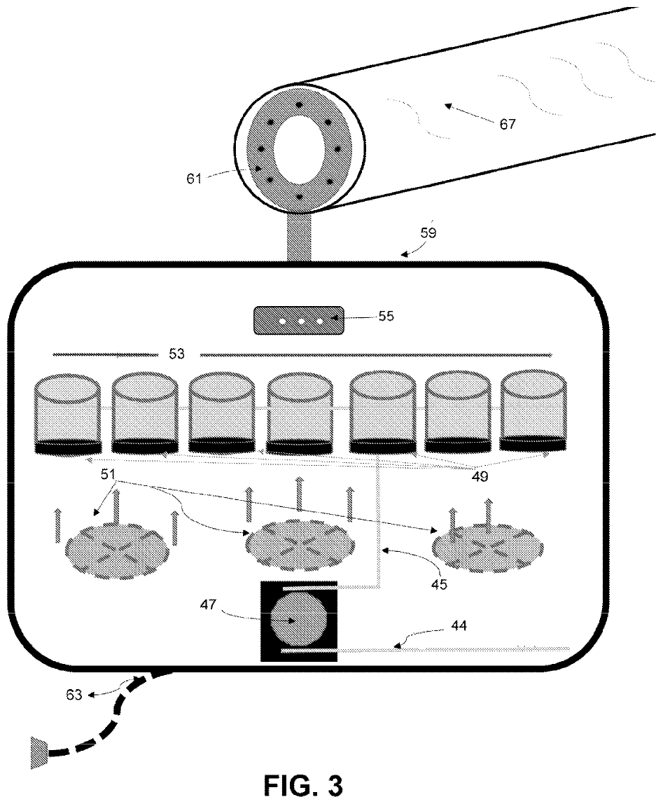

Referring to FIG. 3, a schematic flow diagram illustrates an embodiment of a high flow commercial system. This schematic represents an array of iodine reservoirs and sonic sublimation systems with the gaseous iodine dispersal ring located within the air flow pipe of an HVAC commercial air system.

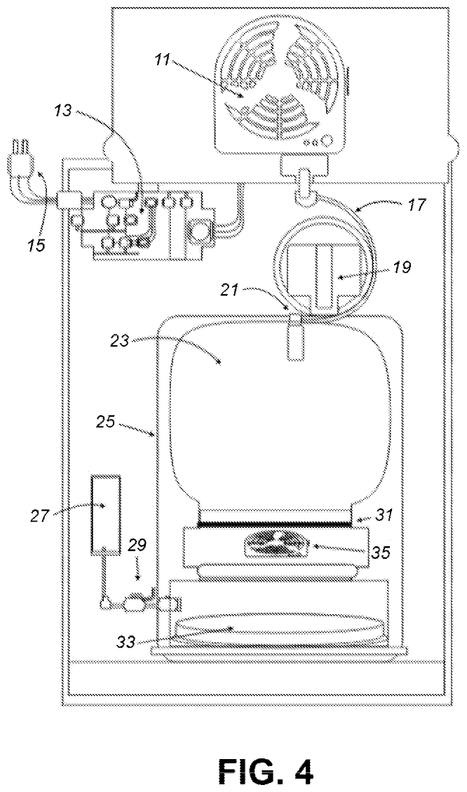

Referring to FIG. 4, in an alternate embodiment of the system, the reservoir receives gaseous iodine from the iodine source. The iodine source includes a cooled iodine dispenser for holding solid iodine and a heat plate for producing gaseous iodine from the solid iodine. A valve connects the iodine dispenser and the heat plate and allows passage of the solid iodine from the dispenser to the heat plate. A heat plate fan located in proximity to the heat plate transports the gaseous iodine from the heat plate to the reservoir. The electronic controller is connected to the valve, for regulating iodine passage from the dispenser to the heat plate.

In an embodiment of the system, the dispersal device comprises a perforated gaseous iodine dispersal ring.

In an embodiment of the system, the reservoir fan moves the iodine from the reservoir to the dispersal device.

In an alternate embodiment of the system, a reservoir peristaltic pump moves the iodine from the reservoir to the dispersal device.

A method for producing atmospheric iodine is provided, including providing iodine from an iodine source to an iodine reservoir, and dispersing the iodine from the reservoir into an atmosphere with an iodine dispersal device, wherein providing and dispersing the iodine is controlled by an electronic connecter.

Atmospheric gaseous iodine is produced through a sublimation process of either liquid or solid iodine from the iodine source. The gaseous iodine is housed in an airless reservoir connected to the iodine source. The sublimation process is controlled by an electronic system capable of monitoring and adjusting the function of devices within the apparatus. Gaseous iodine is directed to the dispersal device within the apparatus by way of positive directional airflow created by a fan. Atmospheric gaseous iodine is introduced in a controlled fashion into a given environment. The invention allows for replaceable mated aqueous or solid iodine recharges to accommodate predetermined maintenance and replenishing schedules of the apparatus

In an embodiment of the method, airless aqueous iodine is provided to the reservoir with an iodine source peristaltic pump. The airless aqueous iodine is sonicated in the reservoir with a sonic frequency disk controlled by the electronic controller, to produce gaseous iodine. Upon the production of the gaseous iodine with the sonic sublimation system, the fan located beneath the reservoir is activated to propel gaseous iodine to the apex of the apparatus through the dispersal device and disseminate the gaseous iodine into the air within a room or establishment.

The sonic system within the invention is frequencied to accelerate the sublimation process of the solid iodine within the aqueous iodine solution thereby achieving controlled sublimation. A known quantity of iodine in an aqueous state is introduced to the heated sonic system reservoir, gaseous iodine is produced by specific sonic frequency emissions passing through the aqueous solution within the reservoir and the resultant gaseous iodine is carried on the air generated by the fan within the apparatus to be dispersed by a perforated ring at the apex of the apparatus into air within a room or establishment. The remaining water in the sonic reservoir post sublimation is then humidified prior to the reservoir's next fill, or sent to drain.

In an alternate embodiment of the method, solid iodine from the iodine source provides gaseous iodine to the reservoir. Solid iodine is passed from a cooled iodine dispenser through a regulated valve to a heat plate to produce gaseous iodine; and the gaseous iodine is transported from the heat plate to the reservoir with a heat plate fan. The passage of the iodine through the valve is regulated by the electronic controller.

Referring to FIG. 4, solid iodine is introduced to a heated non sonic disk 33 to initiate, sustain and control the iodine sublimation process from a solid to gaseous iodine form. In this embodiment, a cooling system is added to maintain the optimal environment for a solid iodine dispensing mechanism 27. Once the iodine gas is produced, the iodine gas then enters an airless reservoir 23, such as an airless teflon or kynar expandable envelop, connected to a peristaltic micro-pump 19 that can accurately meter gaseous iodine from the iodine envelop into an environment or establishment. This embodiment allows for both small and large scale production of gaseous iodine suited for commercial, medical, residential and industrial applications.

In an embodiment of the method, the gaseous iodine is dispersed from the reservoir to the environment through a perforated gaseous iodine dispersal ring.

In another embodiment of the method, the iodine is directed from the reservoir to the dispersal device with a reservoir peristaltic pump.

In another embodiment of the method, the iodine is directed from the reservoir to the dispersal device by way of positive directional airflow created by a fan.

Referring again to FIG. 2, a further object of the invention is it that it may be wall mounted with the apex of the apparatus in a planar fashion located within an air flow return point from a central ventilation system within a room or establishment.

Referring again to FIG. 3, a further object of the invention is interconnecting multiple iodine reservoirs through a liquid feed line, from a remotely located aqueous iodine reservoir to allow for multiple apparatuses drawing from a main aqueous iodine source within a building or an establishment. This accords individual room treatment versus central treatment within a building or establishment.

A further object of the invention is to scale the operation of the individual components within the apparatus to accommodate the production of gaseous iodine for the higher air flows of commercial central treatment systems such as, clinics, hospitals, office buildings and public transit systems including but not limited to trains, ships, planes and automobiles.

Another object of the invention is to provide replaceable iodine solution canisters of predetermined volume and iodine concentration within the apparatus. The iodine canister can include a chemical inert collapsible bladder within the iodine canister. The distinct iodine replacement canister connections are uniquely mated to the connector fittings within the apparatus to ensure the integrity of the system operation.

The present invention has been shown and described in a preferred embodiment. It is recognized, however, that departures may be made within the scope of the invention and that obvious modifications will occur to a person skilled in the art. With respect to the above description, it is to be realized that the optimum dimensional relationships for the parts of the presented invention, to include variations in size, materials, shape, form, function, and manner of operation, assembly and use, are deemed readily apparent and obvious to one skilled in the art, and all equivalent relationships to those illustrated in the drawings and described in the specifications are intended to be encompassed by the present invention.

What is claimed is:

1. A system for producing atmospheric gaseous iodine, comprising:
an airless reservoir for holding an iodine;
an iodine source connected to the reservoir for providing the iodine to the reservoir;
a dispersal device for dispersing the iodine from the reservoir into an atmosphere; and
an electronic controller connected to the iodine source and the dispersal device for controlling the iodine source and the dispersal device.

2. The system of claim 1, wherein
the reservoir receives an airless aqueous iodine and further comprises a sonic frequency disk for sonicating the airless aqueous iodine;
the iodine source comprises an airless aqueous iodine bladder and an iodine source peristaltic pump; and
the electronic controller is further connected to the sonic frequency disk.

3. The system of claim 1, wherein
the reservoir receives a gaseous iodine;
the iodine source comprises
a cooled iodine dispenser for holding solid iodine;
a heat plate for producing gaseous iodine from the solid iodine;
a valve connecting the dispenser and the heat plate to allow passage of the iodine from the dispenser to the heat plate; and
a heat plate fan in proximity to the heat plate for transporting the gaseous iodine from the heat plate to the reservoir; and
the electronic controller is further connected to the valve, for regulating iodine passage from the dispenser to the heat plate.

4. The system of claim 1, wherein the dispersal device comprises a perforated gaseous iodine dispersal ring.

5. The system of claim 1, wherein a reservoir fan moves the iodine from the reservoir to the dispersal.

6. The system of claim 1, wherein a reservoir peristaltic pump moves the iodine from the reservoir to the dispersal device.

7. The system of claim 1, wherein the system is wall mounted and integrates with a central ventilation system.

8. The system of claim 1, wherein the iodine source is located remotely from the iodine reservoir and the dispersal device.

9. The system of claim 1, wherein the iodine source comprises replaceable aqueous iodine canisters.

10. A method for producing atmospheric iodine comprising:
providing an iodine from an iodine source to an iodine reservoir; and
dispersing the iodine from the reservoir into an atmosphere with an iodine dispersal device;
wherein providing and dispersing the iodine is controlled by an electronic connecter.

11. The method of claim 10,
wherein providing the iodine from the iodine source to the iodine reservoir comprises:
providing airless aqueous iodine to the reservoir with an iodine source peristaltic pump; and
further comprising:
sonicating the aqueous iodine in the reservoir with a sonic frequency disk controlled by the electronic controller, to produce gaseous iodine.

12. The method of claim 10,
wherein providing the iodine from the iodine source to the iodine reservoir comprises:
passing solid iodine from a cooled iodine dispenser through a valve to a heat plate to produce gaseous iodine; and
transporting the gaseous iodine from the heat plate to the reservoir with a heat plate fan; and
further wherein the iodine passing through the valve is regulated by the electronic controller.

13. The method of claim 10, wherein dispersing the iodine from the reservoir with an iodine dispersal device comprises dispersing the iodine with a perforated gaseous iodine dispersal ring.

14. The method of claim 10, further comprising moving the iodine from the reservoir to the dispersal device with a reservoir fan.

15. The method of claim 10, further comprising moving the iodine from the reservoir to the dispersal device with a reservoir peristaltic pump.
